(19) United States
(12) Patent Application Publication
(10) Pub. No.: US 2019/0073925 A1
(43) Pub. Date: Mar. 7, 2019

(54) IMAGING PHANTOM AND METHOD OF EVALUATING OPTICAL IMAGING DEVICE

(71) Applicant: OLYMPUS CORPORATION, Tokyo (JP)

(72) Inventors: Yuki SHONO, Tokyo (JP); Ryosuke ITO, Tokyo (JP); Toshiharu NARITA, Tokyo (JP); Hideyuki TAKAOKA, Tokyo (JP)

(73) Assignee: OLYMPUS CORPORATION, Tokyo (JP)

(21) Appl. No.: 16/178,694

(22) Filed: Nov. 2, 2018

Related U.S. Application Data
(63) Continuation of application No. PCT/JP2017/017123, filed on May 1, 2017.

(51) Int. Cl. G09B 23/28; A61B 34/10

(52) U.S. Cl. CPC G09B 23/286; A61B 2034/105; A61B 34/10

(57) ABSTRACT

An imaging phantom according to the present invention includes a main body having an optical characteristic simulating an optical characteristic of biological tissue and a structure (3) installed in the main body, the structure having a fractal structure simulating a tissue structure having a fractal nature present in the biological tissue.

### TECHNICAL FIELD

The present invention relates to an imaging phantom and a method of evaluating an optical imaging device.

### BACKGROUND ART

In the past, in a performance evaluation and demonstration of an optical imaging device for living bodies, phantoms simulating living bodies have been used as a subject. Typical phantoms include two layers having different optical characteristics in order to simulate a layer structure of biological tissue. Other phantoms include a structure of simulating a blood vessel.

### SUMMARY OF INVENTION

### Technical Problem

A tissue structure present in real living bodies has a complicated structure. For example, a blood vessel network in a mucous membrane is formed of many blood vessels having different diameters such as artery and vein, fine artery and vein, and capillary. For example, a doctor recognizes and determines whether tissue is normal or abnormal on the basis of an appearance of a tissue structure from an endoscope image. However, there is no imaging phantom that simulates an appearance of a tissue structure so that an observer can recognize that it looks like biological tissue through a captured image. For example, typical phantoms have no structure of simulating the tissue structure. Other phantoms are too simplified as compared with the real blood vessel network and do not give a feeling of the appearance of the biological tissue sufficiently. Therefore, if typical phantoms, there is a problem in that the performance of the optical imaging device when the real living body is observed is unable to be accurately evaluated on the basis of a more similar appearance when the living body is actually observed.

The present invention was made in light of the foregoing, and it is an object of the present invention to provide an imaging phantom that is capable of realistically simulating the appearance of the tissue structure in the living body using a captured image and a method of evaluating an optical imaging device using the same.

### SOLUTION TO PROBLEM

In order to achieve the above object, the present invention provides the following.

According to a first aspect of the present invention, an imaging phantom includes: a main body having an optical characteristic simulating an optical characteristic of biological tissue; and a structure installed in the main body, the structure having a fractal structure simulating a tissue structure having a fractal nature present in the biological tissue.

According to the first aspect of the present invention, a tissue structure in biological tissue is simulated by a structure, and tissue around the tissue structure is simulated by a main body around the structure. In this case, a captured image in which an appearance of a complicated tissue structure is realistically simulated by the fractal structure of the structure can be obtained.

In the first aspect, the main body may have at least two layers which are stacked, and the at least two layers may be different in at least one of a light scattering property and a light absorption property.

Thus, it is possible to simulate biological tissue including a plurality of layers with at least two layers. Accordingly, it is possible to reproduce an appearance (for example, a color tone and a contrast) similar to real biological tissue.

In the first aspect, the structure may be embedded within at least one of the layers.

Thus, an appearance of the tissue structure (for example, a color tone, a contrast, and/or sharpness) present in the biological tissue can be substantially reproduced by the structure in the layer.

In the first aspect, the tissue structure may be a blood stream structure.

The blood stream structure with the fractal nature is suitable as the tissue structure simulated by the structure.

In the first aspect, the optical characteristic of the main body may simulate an absorption spectrum of blood.

Thus, it is possible to simulate the appearance of the biological tissue more realistically.

In the first aspect, the structure may include a fractal structure with a degree of randomness.

The fractal nature of the tissue structure in the living body has randomness. Therefore, the appearance of the tissue structure can be more realistically simulated by the fractal structure of the structure with the degree of randomness.

In the first aspect, the structure may include a natural object.

Since the fractal structure present in the natural world is used as the structure, it is possible to more realistically simulate the appearance of the fractal structure with degrees of randomness in the living body. For example, in a case in which the natural object is a vein, it is possible to more realistically simulate the bifurcation structure of the blood vessel and the appearance of the stream.

According to a second aspect of the present invention, a method of evaluating an optical imaging device includes photographing the imaging phantom according to the first aspect through the optical imaging device and displaying the acquired image.

According to the second aspect of the present invention, the imaging phantom simulating the optical characteristic of the biological tissue and the fractal structure of the tissue structure is photographed through the optical imaging device, and the same image as when the real biological tissue is observed is obtained. It is possible to accurately evaluate the performance of the optical imaging device when the real biological tissue is observed on the basis of the image.

In the second aspect, the imaging phantom may be irradiated with a plurality of lights having different spectrums, and the plurality of lights may be different in at least one of the light scattering properties and the light absorption properties of the at least two layers.

### Advantageous Effects of Invention

According to the present invention, there is an effect in that it is possible to simulate an appearance of a tissue structure in a living body on the basis of a captured image.

### BRIEF DESCRIPTION OF DRAWINGS

FIG. 1 is an overall configuration diagram of an imaging phantom according to one embodiment of the present invention.

FIG. 2 is a diagram illustrating a structure installed in the imaging phantom of FIG. 1.

FIG. 3 is a view illustrating a cross section of a linear structure taken along lines A-A, B-B, C-C, and D-D of FIG. 2.

FIG. 4 is a diagram illustrating a modified example of the cross section of the linear structure taken along lines A-A, B-B, C-C, and D-D of FIG. 2.

FIG. 5 is a diagram illustrating a modified example of a fractal structure of a structure.

FIG. 6 is a diagram illustrating another modified example of a fractal structure of a structure.

FIG. 7 is a diagram illustrating another modified example of a fractal structure of a structure.

FIG. 8 is a diagram illustrating another modified example of a fractal structure of a structure.

FIG. 9 is a diagram illustrating another modified example of a fractal structure of a structure.

FIG. 10 is a diagram illustrating another modified example of a fractal structure of a structure.

FIG. 11 is a diagram illustrating a modified example of an optical characteristic of a structure.

FIG. 12 is a diagram illustrating a modified example of a fractal structure of a structure having randomness.

FIG. 13 is a diagram illustrating another modified example of a fractal structure of a structure having randomness.

FIG. 14 is a diagram illustrating another modified example of a fractal structure of a structure having randomness.

FIG. 15 is a diagram illustrating another modified example of a fractal structure of a structure having randomness.

FIG. 16 is a diagram illustrating another modified example of a fractal structure of a structure having randomness.

FIG. 17 is a diagram illustrating a modified example of an arrangement of structures.

FIG. 18 is a diagram illustrating another modified example of a main body and a structure.

FIG. 19 is an image of a vein obtained by photographing an imaging phantom of FIG. 18.

FIG. 20 is a diagram illustrating another modified example of an arrangement of structures.

FIG. 21 is a diagram illustrating another modified example of a structure.

FIG. 22 is a view illustrating a cross section of a structure taken along line E-E of FIG. 21.

### DESCRIPTION OF EMBODIMENTS

Hereinafter, an imaging phantom 1 according to one embodiment of the present invention will be described with reference to the appended drawings.

It is noted that the drawings of the present application are provided for illustrative purposes only and, as such, the drawings are not drawn to scale. It is also noted that like and corresponding elements are referred to by like reference numerals.

In the following description, numerous specific details are set forth, such as particular structures, components, materials, dimensions, processing steps and techniques, in order to provide an understanding of the various embodiments of the present application. However, it will be appreciated by one of ordinary skill in the art that the various embodiments of the present application may be practiced without these specific details. In other instances, well-known structures or processing steps have not been described in detail in order to avoid obscuring the present application.

It will be understood that when an element as a layer, region or substrate is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “beneath” or “under” another element, it can be directly beneath or under the other element, or intervening elements may be present . In contrast, when an element is referred to as being “directly beneath” or “directly under” another element, there are no intervening elements present.

In the discussion and claims herein, the term “about” indicates that the value listed may be somewhat altered, as long as the alteration does not result in nonconformance of the process or structure to the illustrated embodiment. For example, for some elements the term “about” can refer to a variation of ±0.10, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either be completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5.4, 5.5, 5.6, 5.7, 5.8, 5.9, etc.

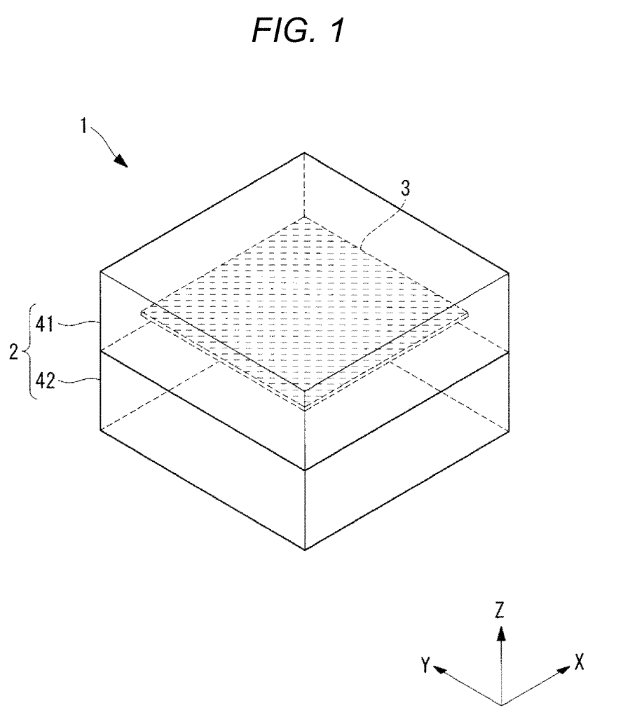

The imaging phantom 1 according to the present embodiment is a phantom simulating biological tissue, and includes a main body 2 and a structure 3 having a structure of simulating a tissue structure present in biological tissue installed in the main body 2 as illustrated in FIG. 1

The main body 2 has a rectangular parallelepiped shape having a traverse direction (an X direction), a longitudinal direction (a Y direction), and a height direction (a Z direction) which are orthogonal to one another and includes two layers 41 and 42 stacked in the height direction. Each of the first layer 41 and the second layer 42 has a uniform thickness. The shape of the main body 2 is not limited to the rectangular parallelepiped shape and may be any other arbitrary shape (for example, a plate shape or a pillar shape).

The first layer 41 and the second layer 42 have the same or similar optical characteristic to or as the layer constituting the biological tissue (for example, the mucous layer or the muscle layer) and have different optical characteristics from each other. The layer structure of biological tissue can be simulated by the two layers 41 and 42.

Specifically, each of the first layer 41 and the second layer 42 has a light scattering coefficient of about 0.1 mm⁻¹ or more and about 5 mm¹ or less in a wavelength range of visible light. Each of the first layer 41 and the second layer 42 has a light absorption property of simulating an absorption spectrum of blood and has a light absorption coefficient of more than about 0 mm¹ and about 20 mm¹ or less in the wavelength range of visible light.

The first layer 41 and the second layer 42 differ in at least one of the light scattering coefficient (light scattering property) and the light absorption coefficient (light absorption property) from each other. The light scattering coefficient of the second layer 42 can be larger than the light scattering coefficient of the first layer 41, or vice versa. The light absorption coefficient of the second layer 42 can be larger than the light absorption coefficient of the first layer 41, or vice versa.

The structure 3 includes a plurality of linear structures which are connected to each other on the same plane and has a relatively thin and substantially flat shape as a whole. The structure 3 is within the first layer 41. The structure 3 is arranged substantially parallel to a surface in the xy plane of the first layer 41 and a surface in the xy plane of the second layer 42 so that a depth from a surface of the first layer 41 on a side opposite to the second layer 42 to the structure 3 is substantially constant. The structure 3 has an optical characteristic different from those of the first layer 41 and the second layer 42.

The structure 3 has a fractal structure of simulating a tissue structure having a fractal nature present in a living body. Examples of the tissue structures having a fractal nature include a blood vessel network, a lung, and a bronchi. In this embodiment, structure 3 can simulate a blood stream structure of a blood vessel network in which bifurcation from one blood vessel to a plurality of thinner blood vessels occurs.

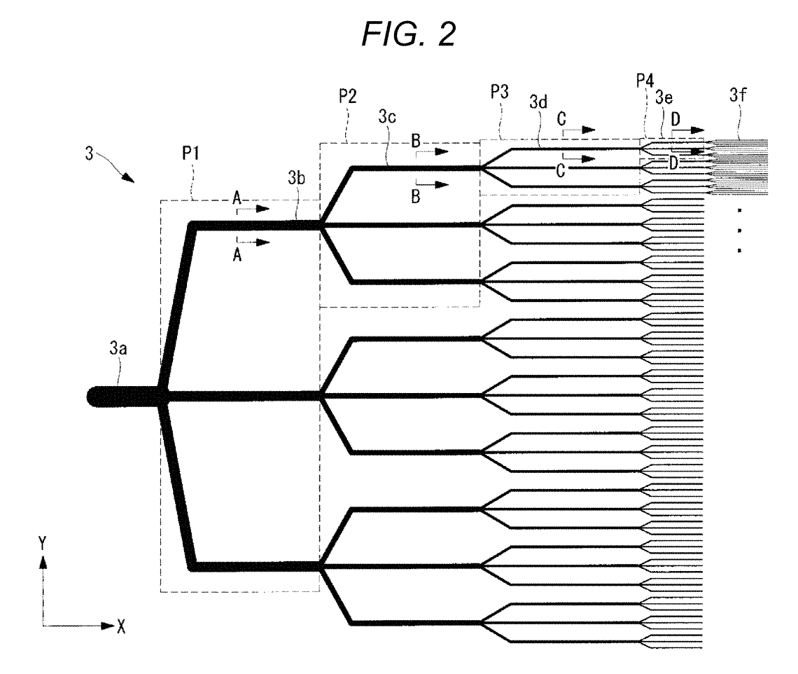

Specifically, as illustrated in FIG. 2, the structure 3 has a structure in which a pattern in which one line is bifurcated into a plurality of lines (three lines in the example illustrated in FIG. 2) at one end (a bifurcation position) is repeated while being reduced in at least one direction. In other words, the structure 3 includes a plurality of patterns P1, P2, P3, and P4 which have a shape including a plurality of lines extending from one bifurcation position and are different in a scale in at least one direction. The shapes of a plurality of patterns P1, P2, P3, and P4 constituting the fractal structure may be analogous to one another or may be similar to one another.

In the patterns P1, P2, P3, and P4 illustrated in FIG. 2, a plurality of lines extending from the bifurcation position are parallel to one another. Further, the patterns P1, P2, and P3 are reduced only in the direction (Y direction) in which intervals of a plurality of lines are narrowed each time bifurcation is performed (as it goes towards the right direction in FIG. 2), and the pattern P4 is reduced in both the X direction and the Y direction.

The structure 3 can be constructed by printing the pattern of the pigments to the transparent sheet. The printed sheet is inserted between the first layer 41 and the second layer 42. At least one of silicon rubber, resin and gel can be used for the transparent sheet. Optical characteristic of the transparent sheet can be aligned with any one of the first layer 41 and the second layer 42.

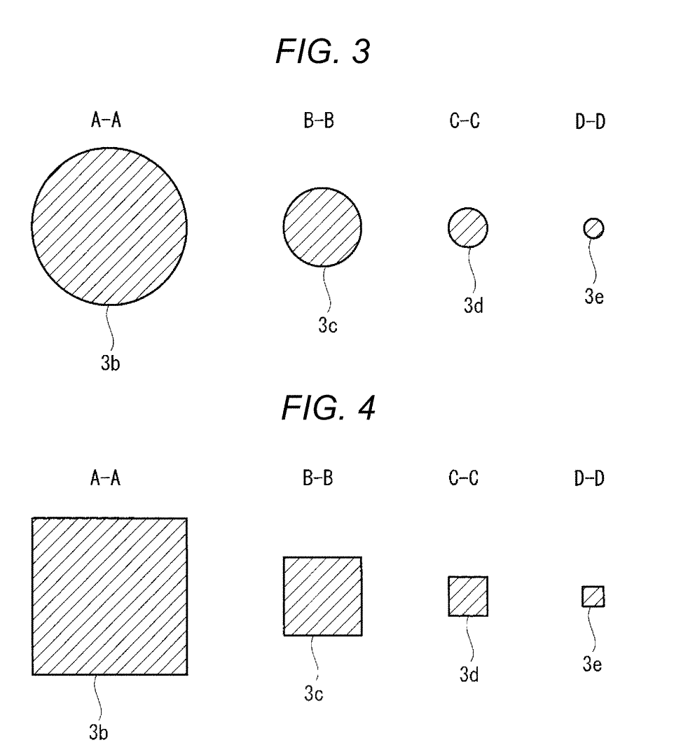

FIG. 3 illustrates cross sections of linear structures 3b, 3c, 3d, and 3e taken along lines A-A, B-B, C-C, and D-D of FIG. 2. As illustrated in FIG. 3, as the scales of the basic patterns P1, P2, P3, and P4 decrease, the width (diameter) and the cross section of the linear structures 3b, 3c, 3d, and 3e are reduced as well. The cross-sectional shapes of the linear structures 3b, 3c, 3d, and 3e may be circular as illustrated in FIG. 3, may be polygonal (for example, quadrangular) as illustrated in FIG. 4, or may be of any shape. Further, all the linear structures 3a, 3b, 3c, 3d, 3e, and 3f can have the same or different cross-sectional shapes as compared to each other.

Next, a method of evaluating an optical imaging device using the imaging phantom 1 having the above configuration will be described.

The imaging phantom 1 according to the present embodiment is used as a subject instead of real biological tissue when an image performance of optical imaging devices for living bodies such as endoscopes is evaluated and demonstrated. The imaging phantom 1 can be arranged such that the first layer 41 is located on an upper side, and the second layer 42 is on a lower side and is observed from the side of the first layer 41 through the optical imaging device. An image (a phantom image) of the imaging phantom 1 acquired through the optical imaging device is displayed on a display. The user can evaluate the image performance of the optical imaging device on the basis of the phantom image displayed on the display.

In this case, the imaging phantom 1 according to the present embodiment has a geometrical structure and optical characteristics which are similar to real biological tissue, and simulates appearances of a tissue structure and tissue around the tissue structure. Therefore, a phantom image that gives recognition as if real biological tissue was being observed is displayed on the display. There is an advantage in that the user can evaluate the image performance of the optical imaging device when real biological tissue is observed on the basis of the phantom image.

Specifically, since the main body 2 including the two layers 41 and 42 having different optical characteristics substantially simulates the optical characteristic of the layer structure of the tissue around the tissue structure simulated by the structure 3, the same color tone and contrast as when the real biological tissue is observed are substantialy reproduced in the phantom image. Particularly, the imaging phantom 1 is photographed through the optical imaging device from the side of the first layer 41 (which in this embodiment has a lower light scattering coefficient and a lower light absorption coefficient as compared to the second layer 42), and thus the same appearance as when the layer structure of the real biological tissue is observed from the surface side is substantially reproduced in the phantom image. Therefore, it is possible to accurately evaluate the image performance, such as the color resolution, of the optical imaging device when the real biological tissue is observed on the basis of the phantom image.

Each of two layers 41 and 42 can be constructed by mixing pigment or dye into the transparent blocks. At least one of silicon rubber, resin and gel can be used for the transparent blocks. Optical characteristic can be controlled by the consistency of the pigment or the dye.

The structure 3 includes a plurality of patterns P1, P2, P3, and P4 in which the linear diameters and the densities of the linear structures 3a, 3b, 3c, 3d, 3e, and 3f are different and has a bifurcation structure in which bifurcation from one linear structure to a plurality of thinner linear structures is repeated. With the structure 3 having such a fractal structure, the appearance of the bifurcation structure of the blood vessel in the real blood vessel network is simulated. Further, since the structure 3 is within the layer 41, the appearance of the tissue structure present inside the biological tissue (for example, the mucous membrane) can be reproduced in the phantom image. Therefore, it is possible to evaluate the image performance, particularly, the spatial resolution of the optical imaging device when the real biological tissue is observed on the basis of the phantom image.

When the phantom image is acquired, the imaging phantom 1 may be irradiated with a plurality of lights having different spectrums . At this time, a spectrum of the light can be selected that is radiated to the imaging phantom 1 so that a plurality of lights are different in at least one of the light scattering properties and the light absorption properties of the layers 41 and 42.

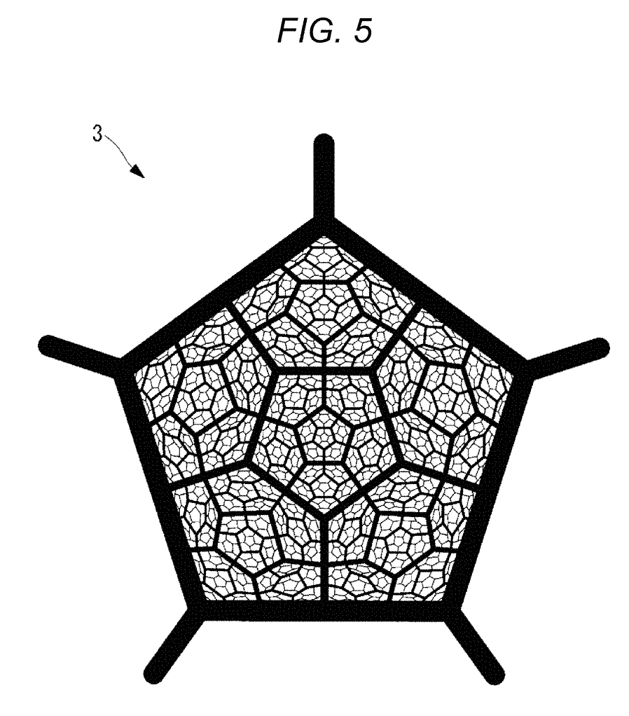

In the present embodiment, the structure 3 has a fractal structure in which the bifurcation from one line to a plurality of lines is repeated but may have any other fractal structure. FIGS. 5 to 8 illustrate modified examples of the fractal structure of the structure 3.

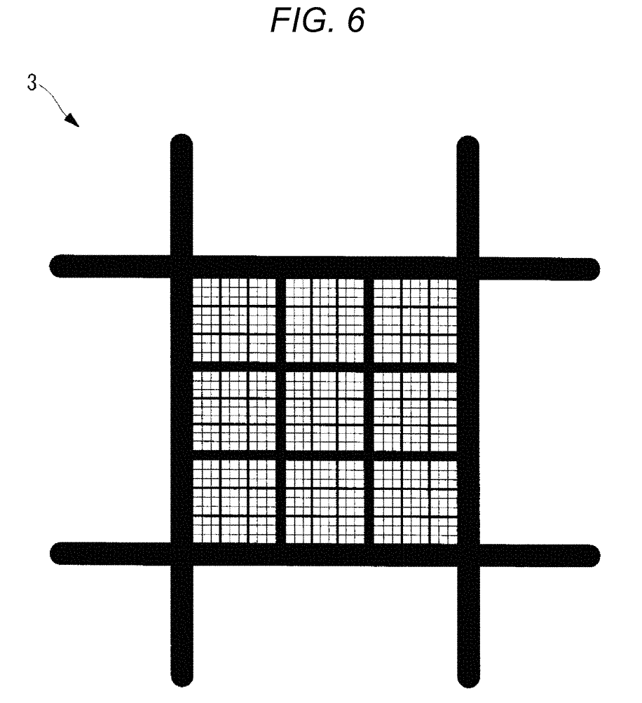

A fractal structure illustrated in FIG. 5 has a pattern including one pentagonal loop line and lines each extending outward from each corner of the loop line, and a reduced pattern is fitted in the loop line. A fractal structure illustrated in FIG. 6 has a pattern including one rectangular loop line and lines in which two lines extend outward from each corner of the loop line, and a reduced pattern is fitted in the loop line. Therefore, the fractal structures of FIGS. 5 and 6 have a bifurcation structure at the corner of the loop line and can simulate the appearance of the feature of the bifurcation structure of the blood vessel. In the outermost pattern, the line extending from the corner may be omitted. In the modified examples, a polygonal loop line other than rectangles and pentagons may be used.

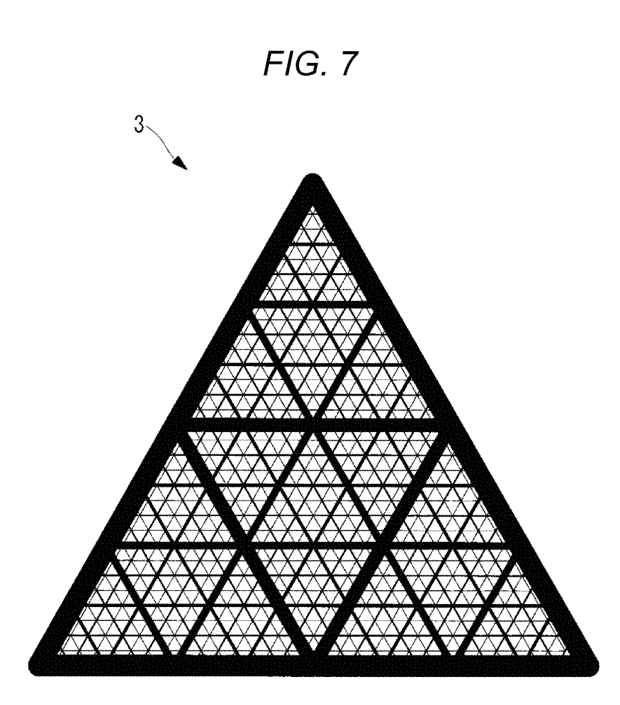

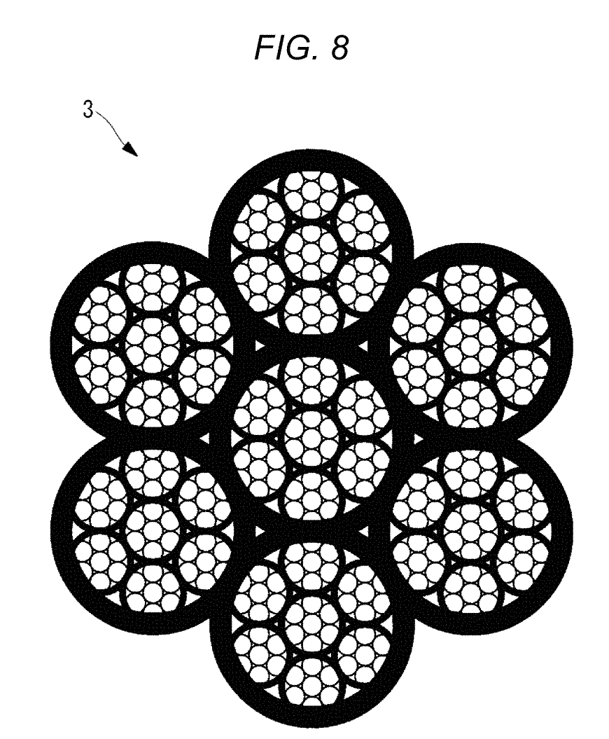

A fractal structure illustrated in FIG. 7 has a pattern including a triangular loop line, and a reduced pattern is rotated by 180° and fitted in the loop line. A fractal structure illustrated in FIG. 8 has a pattern including seven circular loop lines arranged in a hexagonal lattice shape, and a reduced pattern is fitted in each loop line. The fractal structures of FIGS. 7 and 8 can simulate the appearance of a fine pattern (so-called pit pattern) of the mucous membrane.

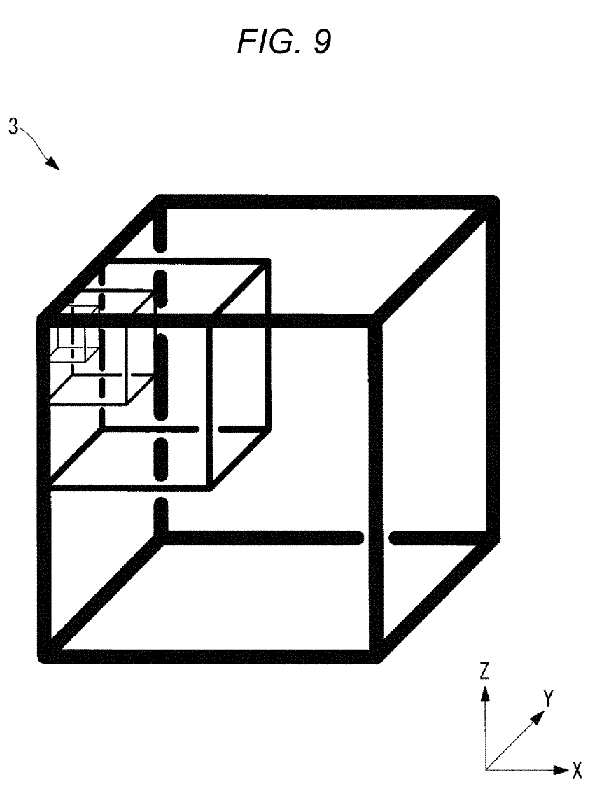

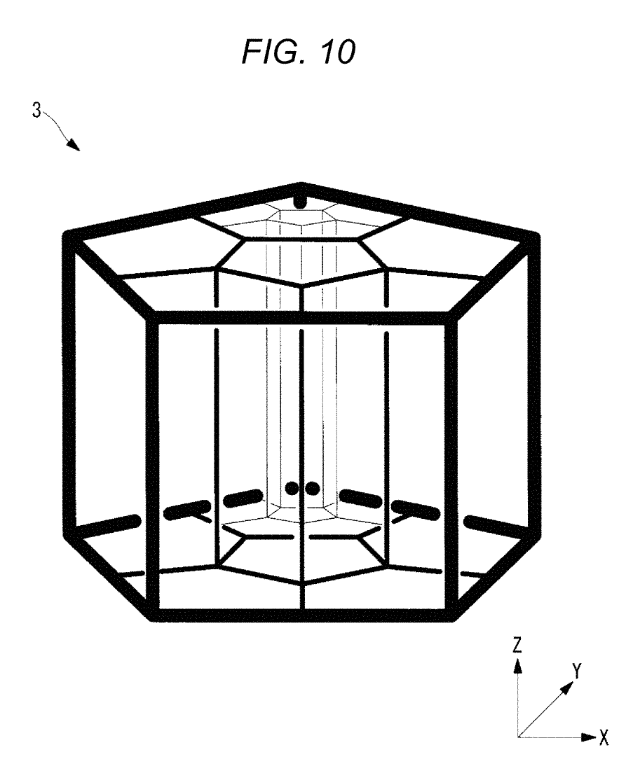

In the present embodiment, the structure 3 has a substantially two-dimensional structure but may have a three-dimensional structure as illustrated in FIGS. 9 and 10. In FIGS. 9 and 10, only a part of the fractal structure is illustrated for simplification of the drawing.

A fractal structure illustrated in FIG. 9 has a pattern including sides of a cube, and a plurality of patterns reduced in the X, Y and Z directions are arranged in the X, Y and Z directions in the cube.

A fractal structure illustrated in FIG. 10 has a pattern including sides of a polygonal column having a longitudinal axis in the Z direction (a pentagonal column in the example illustrated in FIG. 10), and a plurality of patterns reduced in the X and Y directions are arranged in the polygonal column in the X and Y directions.

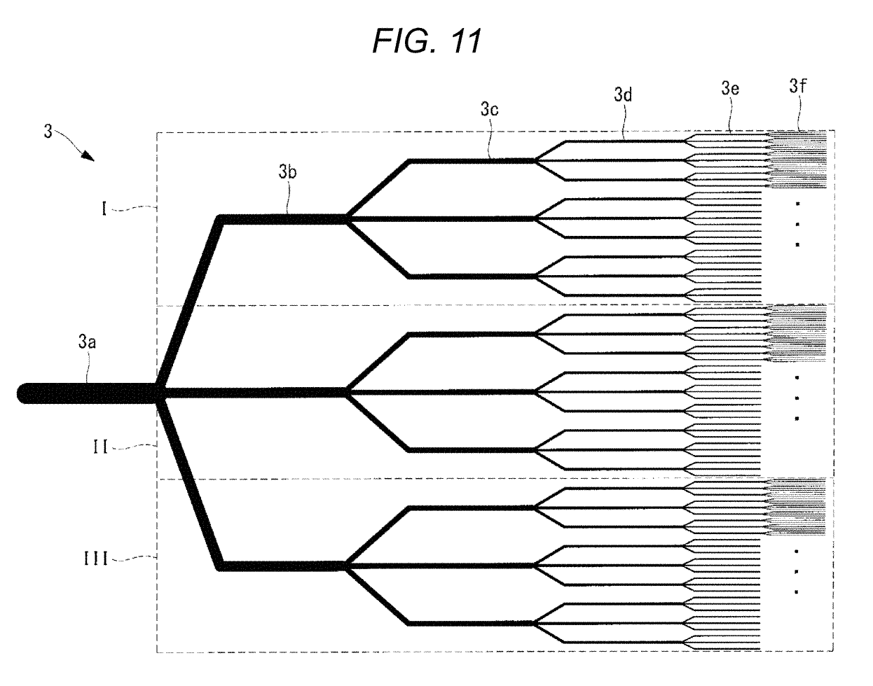

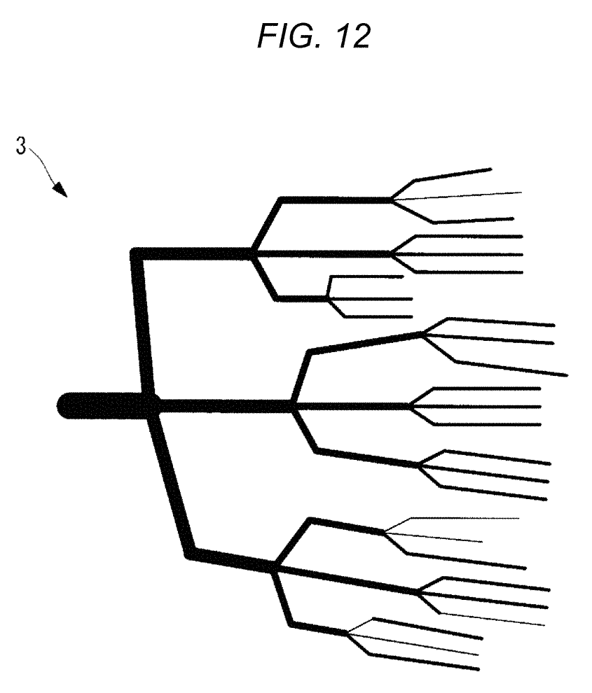

In the present embodiment, the structure 3 has a substantially uniform optical characteristic throughout, but in other embodiments, the structure 3 may be divided into a plurality of compartments I, II, and III as illustrated in FIG. 11. For example, the compartments I, II, and III may have different light absorption coefficients to simulate a difference in blood concentration.

According to the structure 3 of FIG. 11, the image performance (for example, the color resolution) of the optical imaging device for indicating the difference in the appearance between the same type of tissue structures having different optical characteristics can be evaluated on the basis of the phantom image.

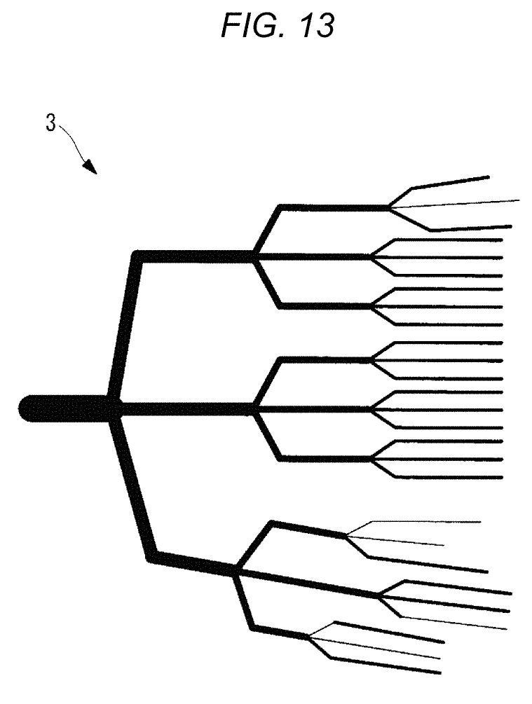

In the present embodiment, the structure 3 has the fractal structure in which structural parameters such as angles, diameters, sizes, and the like of the structures 3a, 3b, 3c, 3d, 3e, and 3f are regularly changed, but instead of this, all or a part of the structure 3 may have a fractal structure with randomness as illustrated in FIGS. 12 and 13. In other words, the angles, the diameters, and the lengths of a plurality of lines extending from the bifurcation position may have a variation within a certain range. In the structure 3 of FIG. 12, the fractal structure has randomness throughout, and in the structure 3 of FIG. 13, a part of the fractal structure has randomness.

The fractal structure present in the living body can have randomness in shape, diameter, direction, size, arrangement, and the like. Since such randomness similar to the fractal structure in the living body can be given to at least a part of the fractal structure of the structure 3, it is possible to simulate the appearance of the tissue structure more realistically.

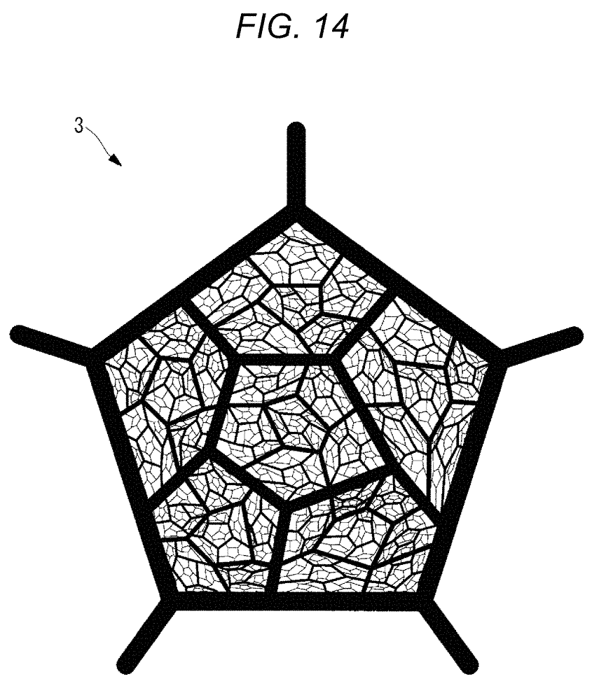

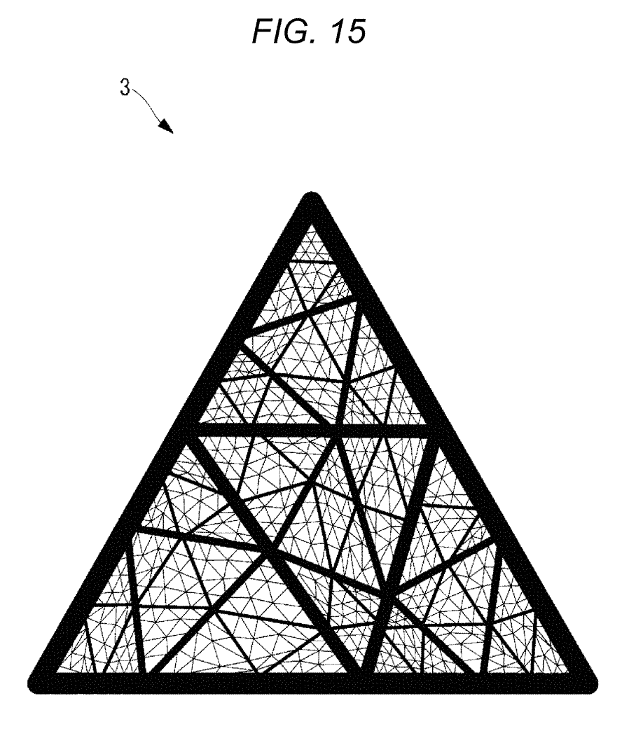

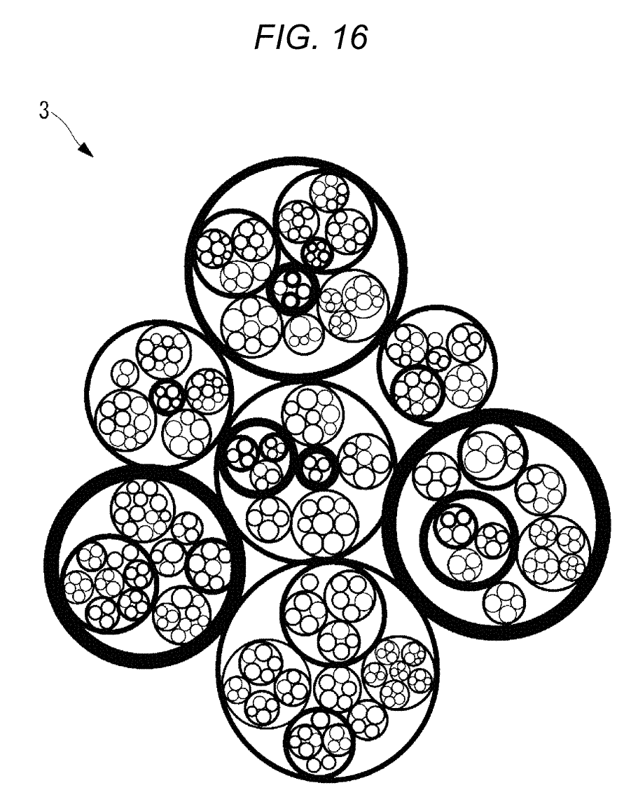

As illustrated in FIGS. 14, 15, and 16, even in fractal structures including a polygonal loop line or a circular loop line, the whole or a part of the fractal structure may have randomness. In other words, the diameter, the shape, the size, and the arrangement of the loop line may have a variation within a certain range.

The fractal structures of the structure 3 illustrated in FIGS. 12 to 16 have randomness in all the structural parameters but may have randomness in only one structural parameter or may have randomness in two or more structural parameters.

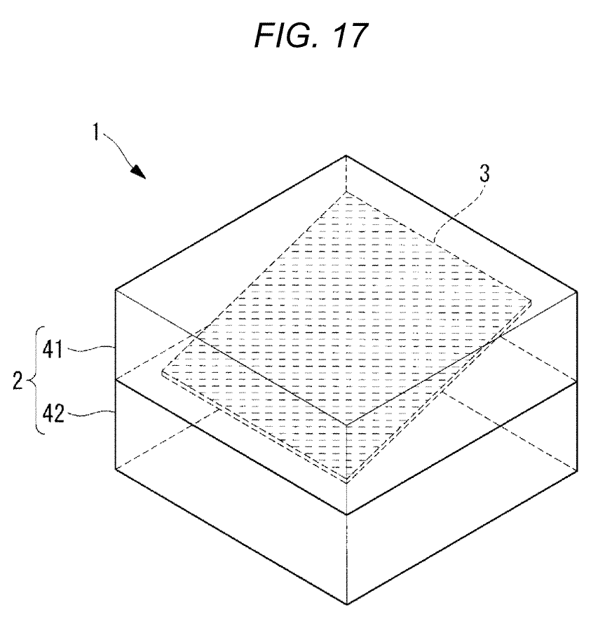

In the present embodiment, the structure 3 is arranged parallel or substantially parallel to the layers 41 and 42 so that the structures 3 are located at substantially the same depth, but in other embodiments, the structure 3 may be obliquely arranged in the layer 41 so that the depth of the structure 3 changes as illustrated in FIG. 17. In the case of the structure 3 of FIG. 2, the structure 3 may be inclined in the X direction, may be inclined in the Y direction, or may be inclined in the z direction, or may be inclined in a combination of x direction and y direction, maybe inclined in a combination of x direction and z direction, maybe inclined in a combination of y direction and z direction, or may be inclined in a combination of x direction, y direction and z direction.

For example, the appearance of the blood vessel in the mucous membrane looks differently depending on the depth in the mucous membrane. According to the imaging phantom 1 of FIG. 17, the image performance (for example, the color resolution) of the optical imaging device for indicating the difference in the appearance between the tissue structures based on the difference in the depth can be evaluated on the basis of the phantom image. Instead of arranging the structure 3 obliquely, in other embodiments a curved structure may be used.

The present embodiment has been described with the created structure 3, but instead of this, a natural object having a fractal structure may be used as the structure 3.

The fractal structure present in the living body has randomness in the shape, the direction, and the scale of the pattern. It is difficult to realistically reproduce an appearance of a structure having such randomness through an design. Since fractal structures existing in the nature world have randomness similarly to the biological tissue, when the natural object is used, it is possible to simulate the appearance of the tissue structure having the fractal nature more realistically.

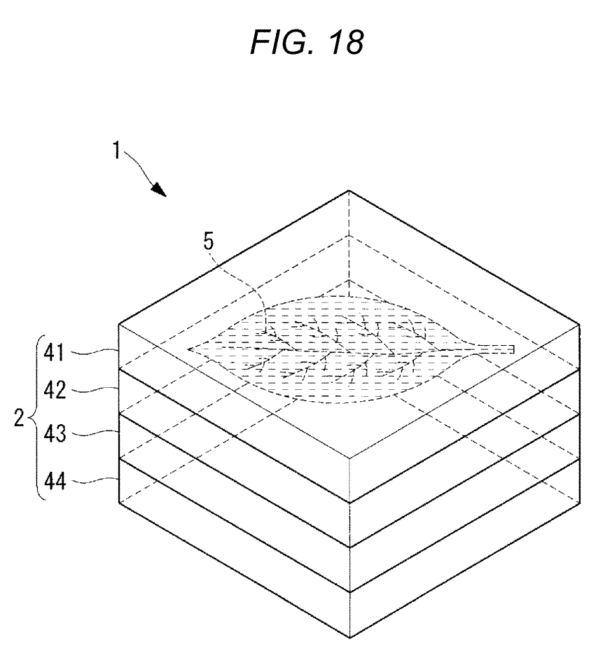

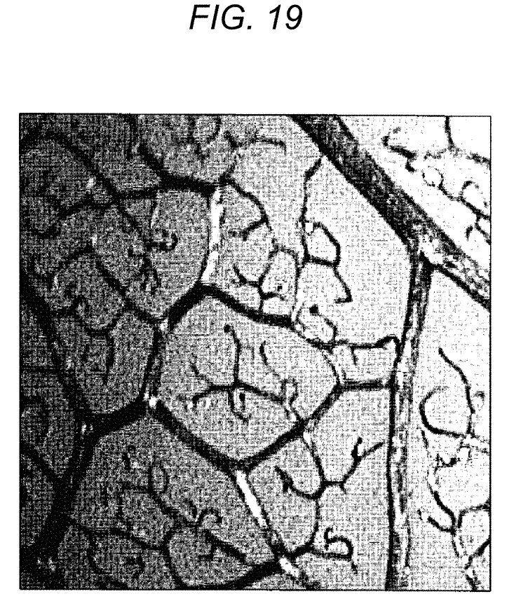

As an example of a natural object, a vein 5 of a natural leaf is illustrated in FIGS. 18 and 19. If the vein 5 is used, it is possible to simulate the bifurcation structure of the blood vessel and the appearance of the stream.

Such an imaging phantom 1 is created by staining the vein 5 with a dye having the same or similar light absorption properties as blood and inserting a leaf having the stained vein 5 in the layer 41. FIG. 19 is an image of the vein 5 obtained by photographing the imaging phantom 1 . The main body 2 of FIG. 18 is an example obtained by simulating a layer structure of a stomach and includes four layers 41 to 44 simulating an epithelium, a lower mucous membrane layer, a muscle layer, and an outer membrane, respectively, in order from one side in the height direction. The leaf is within the layer 41 simulating the epithelium.

In one embodiment, the main body 2includes the two layers 41 and 42, and the structure 3 is within only the first layer 41, but the number of layers and a layer in which the structure 3 are within can be appropriately changed depending on the biological tissue simulated by the imaging phantom 1. For example, the main body 2 may include only one layer or three or more layers, and the structure 3 may be within a layer other than the first layer 41.

Further, the structure 3 may be within a plurality of layers.

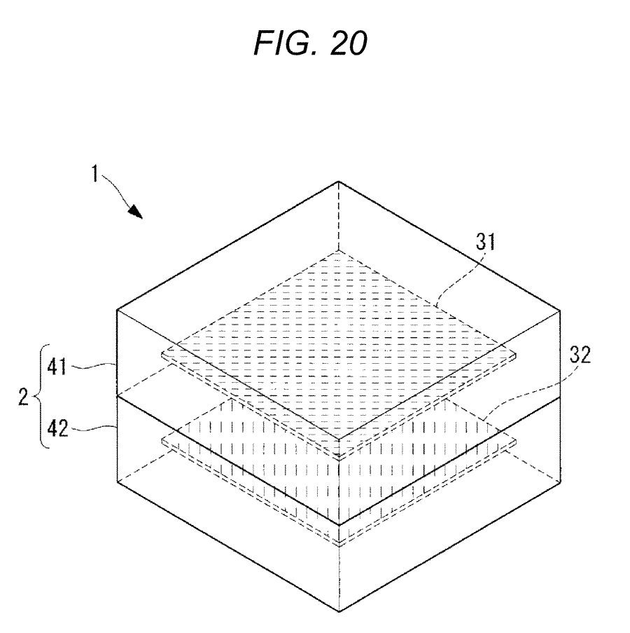

In this embodiment, structures 31 and 32 may be within in each of a plurality of layers 41 and 42 as illustrated in FIG. 20. Further, the structures 31 and 32 in a plurality of layers 41 and 42 may have different fractal structures. In living bodies, generally, the size of the tissue structure increases as the depth of the layer increases. Therefore, the sizes of the fractal structures of the structures 31 and 32 can be in a plurality of layers 41 and 42 and can sequentially increase from the first layer 41 arranged on the upper side toward the second layer 42 arranged on the lower side.

Alternatively, a single structure 3 may be obliquely arranged in the main body 2 to reach a plurality of layers 41 and 42.

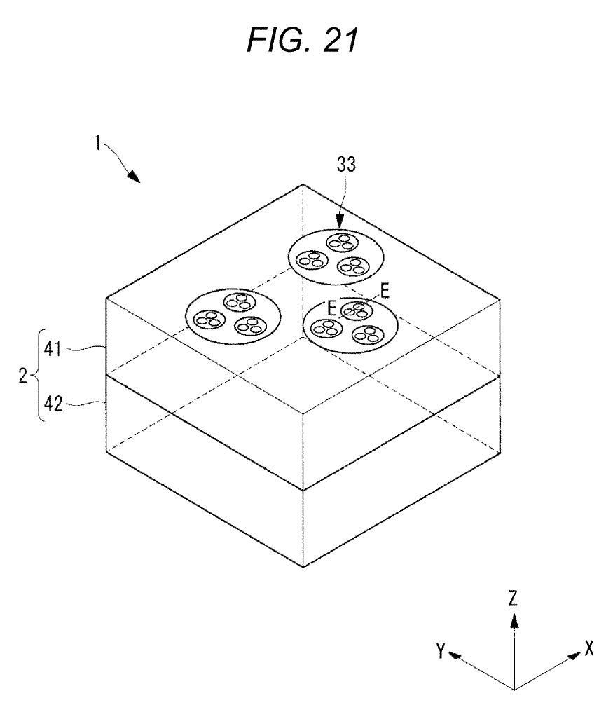

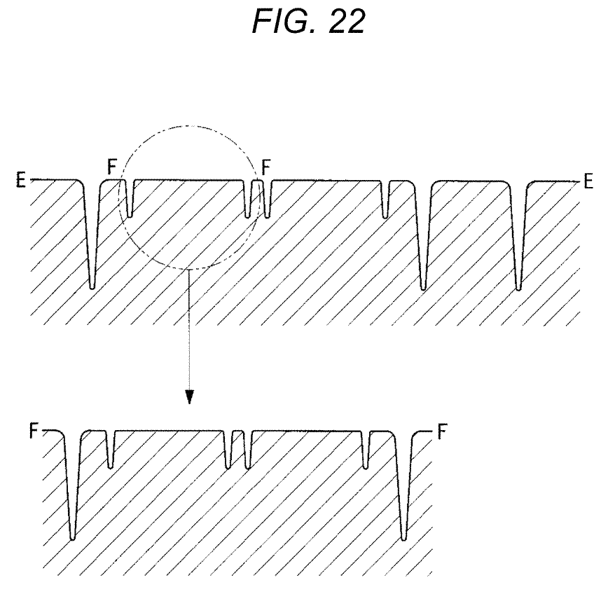

In the present embodiment, the structure 3 is within the main body 2, but instead of or in addition to this, the structure 33 may be formed of a concavo-convex structure having a fractal structure formed on the surface of the main body 2 as illustrated in FIG. 21. FIG. 22 illustrates a cross-sectional shape of the surface of the main body 2 in the structure 33.

The inner wall of the small intestine has a tissue structure with a fractal nature including circular folds and many villi present on the surface of the circular fold. According to the structures 33 of FIGS. 21 and 22, the surface structure of the biological tissue having such fractal nature can be simulated.

The embodiment and the modified examples described above can be appropriately combined and carried out.

### REFERENCE SIGNS LIST

1 Imaging phantom
2 Main body
3, 31, 32, 33 Structure
41, 42, 43, 44 Layer
Vein (structure)

1. An imaging phantom capable of imaging by an optical imaging device,
the imaging phantom comprising:
a main body having a phantom optical characteristic for an observation light, at least one feature of the phantom optical characteristic being the same as the one feature of a original optical characteristic of a biological tissue; and
a structure wthin the phantom body, at least one feature of the structure being same as the one feature of the biological tissue, the structure having a fractal structure.

2. The imaging phantom according to claim 1, wherein the phantom optical characteristic is at least one of a light scattering property and a light absorption property,
the phantom body has a first layer and a second layer stacked on the first layer, the observation light is irradiated form a side of the second layer, and
the phantom optical characteristic of the first layer is different from the phantom optical characteristic of the second layer.

3. The imaging phantom according to claim 2, wherein the phantom structure is embedded within the second layer.

4. The imaging phantom according to claim 1, wherein the biological tissue structure is a blood stream structure,
the original structure is a blood vessel network.

5. The imaging phantom according to claim 1, wherein the original optical characteristic is an absorption spectrum of blood.

6. The imaging phantom according to claim 1, wherein the fractal structure has randomness.

7. The imaging phantom according to claim 1, wherein the phantom structure includes a natural object.

8. The imaging phantom according to claim 7, wherein the natural object is a vein.

9. A method of evaluating the optical imaging device, comprising the steps of: photographing the imaging phantom according to claim 1 through the optical imaging device; and displaying the acquired image.

10. The method of evaluating the optical imaging device according to claim 9, wherein the phantom optical characteristic is at least one of a light scattering property and a light absorption property,
the phantom body has a first layer and a second layer stacked on the first layer,
the observation light includes a first light having a first spectrums, and a second light having a second spectrums,
the phantom optical characteristic of the first layer for the first light is different from the phantom optical characteristic of the second layer for the first light, and
the phantom optical characteristic of the first layer for the second light is different from the phantom optical characteristic of the second layer for the second light.
